(19) United States
(12) Patent Application Publication
(10) Pub. No.: US 2014/0309595 A1
(43) Pub. Date: Oct. 16, 2014

(54) NERVE BLOCKING INDWELLING CATHETER NEEDLE WITH RESISTANCE MEASUREMENT DEVICE

(71) Applicant: Shanghai Tongji Hospital, Shanghai (CN)

(72) Inventors: Bin Yu, Shanghai (CN); Lingjing Jin, Shanghai (CN)

(21) Appl. No.: 14/357,772

(22) PCT Filed: Jul. 9, 2013
(86) PCT No.: PCT/CN2013/000827

§ 371 Date: May 13, 2014

(30) Foreign Application Priority Data
Jul. 23, 2012 (CN) 201210254230.1

(51) Int. Cl. A61B 17/34; A61M 5/48; A61B 19/00; A61M 25/06

(52) U.S. Cl. CPC A61B 17/3401; A61M 25/06; A61M 5/48; A61B 19/46; A61B 2019/464
USPC 604/203

(57) ABSTRACT

A nerve blocking indwelling catheter needle with a resistance measurement device comprises a needle core (6), a catheter (1), and a pressure detection device. The needle core (6) penetrates through the catheter (1), and a tail end of the catheter (1) is connected to a connection tube (19). A rubber plunger (4) is disposed in the connection tube (19). The needle core (6) penetrates through the rubber plunger (4), and a side tube (3) is disposed on a side wall of the connection tube (19) in front of the rubber plunger (4). A channel is disposed in the needle core (6). An opening (5) is disposed on a side wall of the needle core (6) that is located in the connection tube (19) and in front of the rubber plunger (4), and the opening (5) is in communication with the channel. The pressure detection device comprises a shell (20). An inner rod (11) is disposed in the shell (20), and a front end of the inner rod (11) is connected to a rear end of the needle core (6). A pressure sensor (12) is disposed between a tail end surface (13) of the shell (20) and a tail end surface of the inner rod (11), and the pressure sensor (12) is connected to a measurement circuit through a signal wire (17). The measurement circuit is connected to a display device (18). When a needle tip (61) breaks through tunica vaginalis, the pressure on the needle tip (61) has a great difference, and the difference is expressed through the display device (18), so as to prompt operating personnel that the nerve block enters the tunica vaginalis and is adjacent to the nerve to be blocked. The catheter needle is simple and convenient in operation and does a small damage to the nerve.

### FIELD OF THE INVENTION

This invention is related to medical equipment. And more particularly to medical devices, and more particularly to a nerve blocking device with indwelling catheter needle for measuring resistance.

### BACKGROUND

In an operation on a human body, continuous nerve blocking techniques can be implemented to extend the effectiveness of anesthesia, analgesia and/or a sympathetic nerve block. At the same time, continuous nerve blocking techniques can help to administer anesthesia medication at different times at smaller doses thereby reducing the risk of toxicity of local anesthesia. In the prior art, sonography is used independently or together with a nerve stimulator-assisted positioning system to administer the nerve blocking anesthesia. Sonography-guided nerve blocking techniques can be used to visualize the location; although to some extent it reduces complications due to nerve injury, the use of expensive equipment, the requirement for extensive training for the complex operation, and the increase in total number of steps of the operation prohibits easy adoption of the technology. The application of nerve stimulator-assisted positioning technique, on the other hand, often lead to discomfort of a patient due to local muscle twitch. Further the data displayed can help but can not completely prevent the occurrence of a nerve injury. At present, a paresthesia positioning method is commonly used to determine the position of the nerve block. Because the possibility of a nerve injury, patients sensing shock and discomfort, and because of the differences in nerve distribution and condition of patients, the patient may not be be to sense the paresthesia.

### SUMMARY OF THE INVENTION

One object of the present invention is to provide an apparatus comprising a nerve blocking indewelling catheter needle with a block resistance measurement; such a measuring device having a nerve blocking indewelling catheter needle can solve the technical problems of current nerve block positioning technique that suffer complex or unreliable operation and patient discomfort.

This present invention discloses a resistance measurement device of a nerve blocking indewelling catheter needle, comprising a needle core, a catheter and a pressure detection device. The needle core penetrates through the catheter, wherein the front end of the needle core is provided with a needle tip, the needle tip extends outside of the outer surface of the front end of the catheter. At least one through hole is disposed on the side wall of the front end of the catheter. A tail end of the catheter is fixed to a connection tube, and a jacket tube is disposed on the periphery of the junction where catheter tail end and the connection tube meet. A rubber plunger is disposed inside the connection tube. The outer periphery of the rubber plunger seals with the inner wall of the connection tube. The needle core penetrates through the rubber plunger. A side tube is disposed on a sidewall of the connection tube in front of the rubber plunger. The side tube is in communication with the connection tube. A channel is disposed in the needle core. An opening is disposed on a sidewall of the needle core that is located in the connection tube and in front of the rubber plunger, and the opening is in communication with the channel. The front end of the channel opens at the needle tip. The pressure detection device comprises a housing. An inner rod is disposed in the housing. A pair of axial slides is disposed between the inner rod and the housing. A tail end surface of the housing is enclosed. A pressure sensor is disposed between a tail end surface of the housing and a tail end surface of the inner rod, and the pressure sensor is connected to a signal wire. The signal wire is further connected to a measurement circuit. The measurement circuit is connected to a display device or audio presentation device. The front end of the inner rod is connected to the tail end of the needle core.

Further, the front end of the inner rod is connected to the tail end of the needle core via a connecting member. Said connecting member is disposed in the connection tube. A pair of slides is disposed between the connecting member and the connection tube. The front end of the connecting member and the tail end of the needle core is fixed. An axial blind hole is disposed in the connecting member. Said axial blind hole opens in the end surface of the connecting member. The front end of the inner rod extends into the axial blind hole.

Further, a long slot is disposed, in parallel to the axial length of the housing, in the sidewall of the housing.

A radial projecting lever is disposed on the inner rod. Said radial projecting lever penetrates through said long slot.

Further, a spring is disposed as a cap to the inner rod in front of the radial projecting lever.

Further, a button is disposed on the outside of the radial projecting lever.

Further, a handle is fixed on the outside of the housing.

Further, the central axes of the connecting tube and the catheter extension line coincide.

Further, an X-ray shielding line is disposed in the inner wall of the catheter. The X-ray shielding line extends in the longitudinal direction of the catheter.

Further, the outer surface of the needle core is provided with a scale.

Further, the needle tip comprises a slope. The slope and the axis of the needle core form an angle of 45 degrees.

In the present invention, the pressure sensor, the measurement circuit for the pressure signal, the display device and audio presentation device all adopt readily known technologies in the art. The technology concerning pressure sensors, pressure signal measurement circuits, display devices and audio presentation devices are all known to one skilled in the art, therefore the detail implementation is not recited herein.

The present invention works based on the following principles. The human body anatomy shows that most of the nerve is located underneath a sheath or fascia. In clinical practice, when blocking the nerve by breaking through the sheath, different resistance is felt by hand when a needle tip goes through layers of tissues. Along the path of the needle, while the needle tip goes through different layers of tissues, breaking into sheath or fascia, besides skin, there is the most evident resistance change.

Therefore, before the the nerve blocking operation, an operator first to determine the precise position of the nerve by anantomy. After applying local anesthesia, the operator holds the housing and inserts the needle tip into the subcutaneous tissue along anatomical pathways. Back pressure on the needle is passed on to the inner rod, the inner rod passes the sensed pressure to the pressure sensor, the pressure sensor transmits the pressure signal in real time to the measurement circuit, the pressure signal measurement circuit displays the pressure values sensed by the needle tip by a display or audio presentation device. As the needle breaks through sheath or fascia, the needle will sense an enormous pressure drop, the values of the pressure drop is expressed by a display or audio presentation device to alert an operator that the needle has entered underneath the sheath or fascia, near the nerves to be blocked. A standard value for the pressure drop can be determined by prior experiments. When the measured pressure drop exceeds the standard value, a decision can be made. If at the same time of the pressure drop, no blood is back flowed into the connection tube in front of the rubber plunger, 3-5 mL of local anthestic can be administered through the side tube. The local anesthetic is injected into tissue by passing it through the opening on the sidewall of the needle core, the channel in the needle core and the opening inside the needle tip. Then the jacket tube is held by hand, the catheter is inserted slowly into the tissue for about 3 cm. The needle core is then subsequntly removed by holding the housing and pulling backwards and the catheter remains in the desired area. The remaining drug solution is injected to the desired area continuously through the side tube. The analgesia pump is connected after administering the anesthesia. After the removal of needle core, the rubber plunger in the connection tube seals the connection tube automatically to prevent over-flow of the liquid. Since through holes are disposed on the sidewall of the front portion of the catheter, even if the front opening of catheter is plugged by some soft tissue, the drug solution can still be continuously infused into the patient. The x-ray shielding wire inside the catheter can help to locate the catheter and be used to observe the injection status under X-rays.

The slope of the needle tip and the axial direction of the needle core form an angle of 45 degrees. By using a circular section of the needle tip, it makes it easier to sense resistance when breaking into the sheath or fascia. Because the needle core is not sharp, it does not damage nerves easily. Clinical trials have proven that if the positioning with respect to the nerve before the operation is accurate, the method disclosed in the present invention is easy to adopt and introduces less possibility of nerve damage. When the nerve is blocked, the resistance of different tissues can clearly indicate the position of the needle, especially when the needle enters into the sheath or fascia. The present invention simplifies the steps in an operation and avoids nerve damage, and can also save the cost of buying expensive ultrasound equipment. The present invention does not cause discomfort to patients due to the stimulated muscle spasms. And the present invention can be used in patients during sedation or recovery; therefore it has important clinical significance.

Comparing the present invention with the prior art, the improvement effect is positive and significant. In the present invention, a pressure sensor is disposed at the tail end of the needle core. The signal from the pressure sensor is connected to a measurement circuit and sent to a display device. When the needle tip progresses along anatomical pathways, the display device displays the pressure value sensed by the needle tip in real time. When an enormous pressure drop is detected by the pressure sensor, the operator is alerted that the needle tip has entered under the sheath or fascia, and is near the nerve to be blocked. In the present invention, a through hole is disposed on the side wall of the front portion of the catheter. Even if the front opening of the needle tip is blocked by soft tissue, the through hole can still ensure continuous infusion of the drug solution. The slope of the needle tip and the axial direction of the needle core form an angle of 45 degrees, together with a circular section for the needle tip, which facilitates sensing of the resistance. At the same time, the needle core is not sharp so as to minimize nerve damage. The present invention simplifies the steps in an operation and avoids nerve damage, and can also save the cost of buying expensive ultrasound equipment. The present invention does not cause discomfort in patient due to stimulated muscle spasms. And the present invention can be used in patients during sedation or recovery; therefore it has important clinical significance.

### BRIEF DESCRIPTION

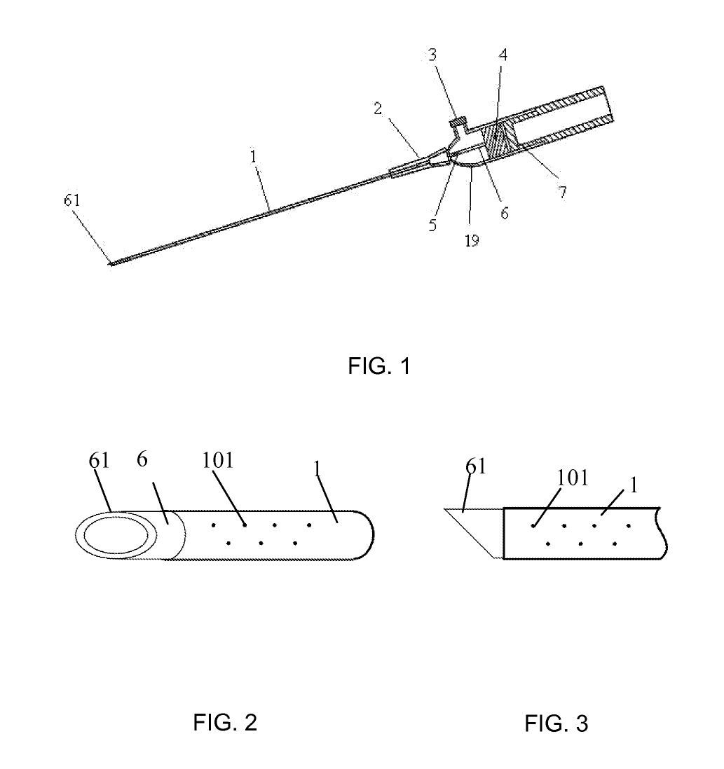

FIG. 1 is a schematic illustration of the needle core and the catheter of the present invention.

FIG. 2 is a schematic illustration of the needle tip and the front of catheter in the present invention.

FIG. 3 is a schematic illustration of the needle tip and the front of catheter view in another direction.

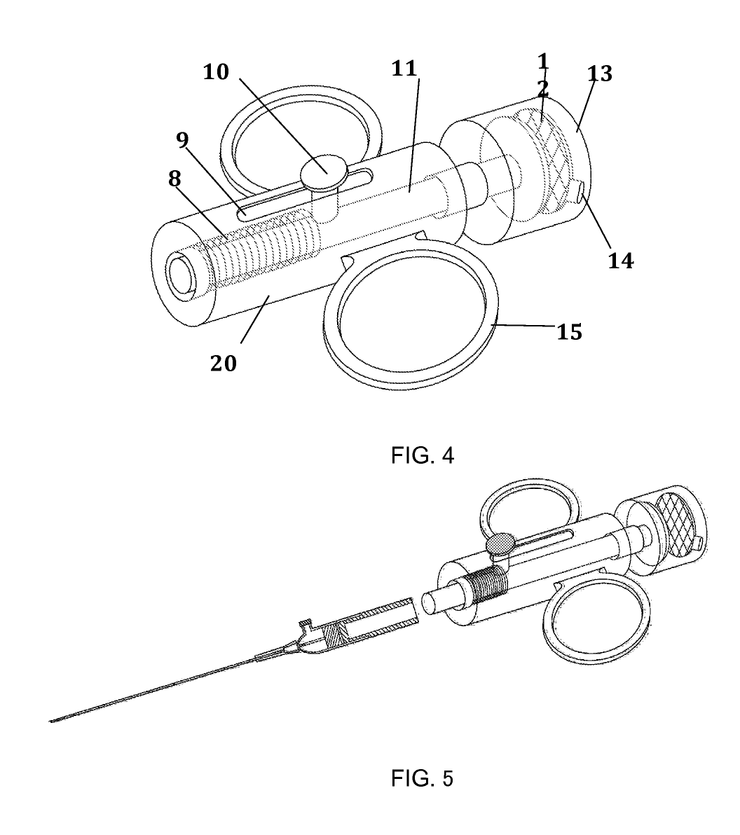

FIG. 4 is a schematic illustration of the pressure detecting device of the present invention.

FIG. 5 is a schematic illustration of the structure of the nerve blocking indewelling catheter needle with resistant measurement device of the present invention.

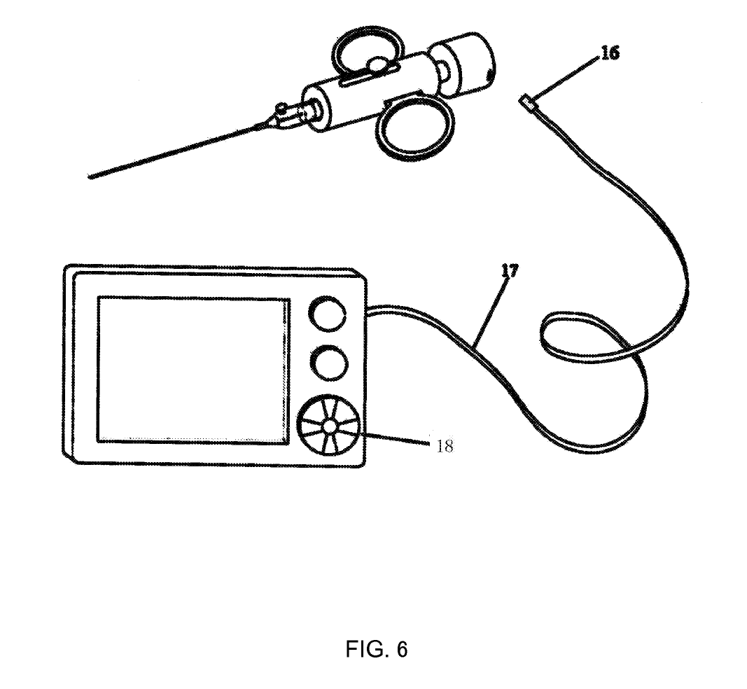

FIG. 6 is a schematic illustration of the structure of the nerve blocking indewelling catheter needle with resistant measurement device of the present invention, which comprises a signal wire and display device.

### SPECIFIC EMBODIMENT

### EXAMPLE 1

Referring to FIG. 1, FIG. 2, FIG. 3, FIG. 4, FIG. 5, and FIG. 6, the present invention discloses nerve blocking indwelling catheter needle having a resistence measurement device, comprising a needle core 6, a catheter 1 and a pressure detecting device, wherein the needle core 6 penentrates through the catheter 1. The front end of the needle core 6 is provided with a needle tip 61, the needle tip 61 extends outside of the outer surface of the front end of the catheter 1. At least one through hole 101 is disposed on the side wall of the front end of the catheter 1. A tail end of the catheter 1 is fixed to a connection tube 19, and a jacket tube 2 is disposed on the periphery of the junction where catherter tail end and the connection tube meet. A rubber plunger 4 is disposed inside the connection tube 19. The outer periphery of the rubber plunger 4 seals with the inner wall of the connection tube 19. The needle core 6 penetrates through the rubber plunger 4. A side tube 3 is disposed on a sidewall of the connection tube 19 in front of the rubber plunger 4. The side tube 3 is in communication with the connection tube 19. A channel is disposed in the needle core 6. An opening (5) is disposed on a sidewall of the needle core (6) that is located in the connection tube (19) and in front of the rubber plunger (4), and the opening (5) is in communication with the channel. The front end of the channel opens at the needle tip 61. The pressure detection device comprises a housing 20. An inner rod 11 is disposed in the housing 20. A pair of axial slides is disposed between the inner rod 11 and the housing 20. A tail end surface of the housing 20 is enclosed. A pressure sensor (12) is disposed between a tail end surface (13) of the housing (20) and a tail end surface of the inner rod (11), and the pressure sensor (12) is connected to a signal wire (17). The signal wire 17 is further connected to a measurement circuit (not shown). The measurement circuit is connected to a display device 18. The front end of the inner rod 11 is connected to the tail end of the needle core 6.

Further, the housing 20 is provided with a port 14 connected to a signal wire, there is provided a signal wire 17 connected to pin 16, the signal wire connected to the connecting port 14 is connected with the plug 16.

Further, a long slot 9 is disposed, in parallel to the axial length of the housing 20, in the sidewall of the housing. An radial projecting lever is disposed on the inner rod 11. Said radial projecting lever penetrates through said long slot 9.

Further, a spring 8 is disposed as a cap to the inner rod 11 in front of the radial projecting lever.

Further, a button 10 is disposed on the outside of the radial projecting lever.

Further, a handle 15 is fixed on the outside of the housing 20.

Further, the central axies of the connecting tube 19 and the catheter 1 extension line coincide.

Further, an X-ray shielding line (not shown) is disposed in the inner wall of the catheter 1. The X-ray shielding line extends in the longitudinal direction of the catheter.

Further, the outer surface of the needle core 6 is provided with a scale.

Further, the needle tip 61 comprises a slope. The slope and the axie of the needle core 6 form an angle of 45 degrees.

The present invention works based on the following principles. The human body anatomy shows that most of the nerves locate underneath the sheath or the fascia. In clinical practice, when blocking a nerve by breaking through a sheath, different resistance is felt by hand when the needle tip 61 goes through different layers of tissues. Along the path of the needle, when needle tip going through different layer of tissues, breaking into sheath or fascia, besides skin, there is the most evident resistance change. Therefore, before the nerve blocking operation, an operator first determine the precise position of the nerve using anatomy. After applying local anesthenigia, an operator can insert the needle tip 61 into the subcutaneous tissue along anatomical pathways by holding the housing 20. Back pressure on the needle tip 61 is passed on to the inner rod 11, the inner rod 11 passes the sensed pressure from needle tip 61 to the pressure sensor 12, the pressure sensor transmits the pressure signal in real time to the measurement circuit, the pressure signal measurement circuit displays the pressure values sensed by the needle tip by a display 18 or audio presentation device, alerting the operator as the needle breaks through a sheath or fascia, the needle senses an enormous pressure drop, the values of the pressure drop is expressed by a display or audio presentation device to alert the operator that the needle tip has entered underneath the sheath or fascia, near the nerves to be blocked. A standard value for the pressure drop can be determined by prior experiments before the operation. When a measured pressure drop exceeds the standard value, a decision can be made. If at the same time of the pressure drop, no blood is back flowed into the connection tube 19 in front of the rubber plunger 4, 3-5 mL of local anthestic can be administered through the side tube 3. The local anesthetic is injected into the tissues by passing through the opening 5 on the sidewall of the needle core 6, the channel in the needle core 6 and the opening inside the needle tip 6. Then the jacket tube 2 is held by hand, the catherter is inserted slowly into the tissue for about 3 cm. The needle core 6 is then subsequently removed by holding the housing 20 and pulling backwards leaving the catheter in the desired area. The remaining drug solution is injected to the desired area continuously through the side tube 3. The analgesia pump is connected after the anesthesia operation. After the removal of needle core 6, the rubber plunger 4 in the connection tube 19 seals the connection tube 19 automatically to prevent the overflow of the drug solution. Since through holes 10 are disposed on the sidewall of the front portion of the catheter 1, even if the front opening of catheter 1 is plugged by some soft tissue, the drug solution can still be continuously infused into the patient. The x-ray shielding line inside the catheter can help to locate the catheter and observe the injection status under X-rays.

The slope of the needle tip 61 and the axial direction of the needle core 6 form an angle of 45 degrees. By using a circular section of the needle tip 6, it makes it easier to sense resistance when it breaks into the sheath or fascia. Because the needle core 6 is not sharp, it minimize the possibility of nerve damage. Clinical trials have proven that if the positioning for the nerve before the anethesia operation is accurate, the method disclosed in the present invention is easy to adopt and introduces less possibility of nerve damage. When a nerve is blocked, the resistance of different tissues can clearly indicate the position of the needle, especially when the needle enters into the sheath or fascia. The present invention simplifies the steps in an operation and avoids nerve damage, and can also save the cost of buying expensive ultrasound equipments. The present invention does not cause discomfort in patient due to the stimulated muscle spasms. And the present invention can be used in patients during sedation or recovery, therefore it has important clinical significance.

1. A nerve blocking indewelling catheter needle having a resistance measurement device, comprising a needle core, a catheter and a pressure measurement device, wherein: said core penetrates through said catheter, the needle core penetrates through the catheter, wherein the front end of the needle core is provided with a needle tip, the needle tip extends outside of the outer surface of the front end of the catheter, at least one through hole is disposed on the side wall of the front end of the catheter, a tail end of the catheter is fixed to a connection tube, and a jacket tube is disposed on the periphery of the junction where catherter tail end and the connection tube meet, wherein a rubber plunger is disposed inside the connection tube, the outer periphery of the rubber plunger seals with the inner wall of the connection tube, the needle core penetrates through the rubber plunger, wherein a side tube is disposed on a sidewall of the connection tube in front of the rubber plunger, and the side tube is in communication with the connection tube, wherein a channel is disposed in the needle core, wherein an opening is disposed on a sidewall of the needle core that is located in the connection tube and in front of the rubber plunger, and the opening is in communication with the channel, wherein the front end of the channel opens at the needle tip, wherein the pressure detection device comprises a housing, wherein an inner rod is disposed in the housing, and a pair of axial slides is disposed between the inner rod and the housing, wherein a tail end surface of the housing is enclosed, wherein a pressure sensor is disposed between a tail end surface of the housing and a tail end surface of the inner rod, and the pressure sensor is connected to a signal line, wherein the signal line is further connected to a measurement circuit, and the measurement circuit is connected to a display device or audio presentation device, wherein the he front end of the inner rod is connected to the tail end of the needle core, wherein a long slot is disposed, in parallel to the axial length of the housing, in the sidewall of the housing, wherein a radial projecting lever is disposed on the inner rod and said radial projecting lever penetrates through said long slot. A spring is disposed as a cap to the inner rod in front of the radially projecting lever, wherein a button is disposed on the outside of the radial projecting lever.

2. The nerve blocking indewelling catheter needle of claim 1, characterized in that: the front end of the inner rod is connected to the tail end of the needle core via a connecting member and said connecting member is disposed in the connection tube wherein a pair of slides is disposed between the connecting member and the connection tube wherein the front end of the connecting member and the tail end of the needle core is fixed wherein an axial blind hole is disposed in the connecting member said axial blind hole opens in the end surface of the connecting member and the front end of the inner rod extends into the axial blind hole.

3. The nerve blocking indewelling catheter needle of claim 1, characterized in that: said outer housing is provided with a fixed handle.

4. The nerve blocking indewelling catheter needle of claim 1, characterized in that: the central axies of the connecting tube and the catheter extension line coincide.

5. The nerve blocking indewelling catheter needle of claim 1, characterized in that: X-ray shielding line is disposed in the inner wall of the catheter and the X-ray shielding line extends in the longitudinal direction of the catheter.

6. The nerve blocking indewelling catheter needle of claim 1, wherein: the outer surface of the needle core is provided with a scale.

7. The nerve blocking indewelling catheter needle of claim 1, characterized in that: the needle tip comprises a slope and the slope and the axie of the needle core form an angle of 45 degrees.
